(19) United States
(12) Patent Application Publication
(10) Pub. No.: US 2010/0172947 A1
(43) Pub. Date: Jul. 8, 2010

(54) Pediculicidal Process And Composition

(75) Inventors: Nicolas Palangie, Le Meux (FR); Jean-Philippe Pascal, Nancy (FR)

Correspondence Address: Solvay;c/o B. Ortego - IAM-NAFTA, 3333 Richmond Avenue, Houston, TX, 77098-3099, US

(73) Assignee: SOLVAY SA

(21) Appl. No.: 12/090,444

(22) PCT Filed: Oct. 13, 2006
(86) PCT No.: PCT/EP2006/067367

§ 371 Date: Apr. 16, 2008

(30) Foreign Application Priority Data
Oct. 17, 2005 (FR) 05.10553

(51) Int. Cl. A61K 33/00; A01N 25/12; A01N 59/00; A61P 33/14; A01P 7/04

(52) U.S. Cl. 424/405; 424/717

(57) ABSTRACT

Pediculicidal process which make it possible to combat the growth of the louse Pediculus humanus, according to which the louse is brought into contact with a composition devoid of neurotoxic compounds comprising at least 60% by weight of alkali metal bicarbonate.

The invention relates to a pediculicidal process and to a pediculicidal composition, that is to say a process and a composition which are effective in combating the arthropod Pediculus humanus and/or its nits. It also relates to a method for killing the Pediculus humanus arthropods growing on human head hair using such a composition.

The lice growing on the human body (Pediculus humanus) present a known public health problem. These lice comprise, in a known way, Pediculus humanus capitis (head louse), indeed even Pediculus humanus corporis (body louse), and Phthirus pubis (pubic louse). Although the infestation of children by lice has been declining in recent decades, in France alone more than two million children still have to be treated each year, which is a worry to families and officials of primary schools concerned. The struggle against lice is made particularly complex in that the eggs of lice are protected by an extremely watertight casing and are firmly attached to human hair. This casing exhibits only a few very small openings, known as aeropiles, which are situated on an operculum at the top of the nit, allowing the nit to breathe, these aeropiles constituting the only passages which allow a pediculicide to penetrate into the nit. During the hatching of the nits, the operculum comprising the aeropiles opens to allow the nymph, which climbs out of the watertight protective casing, to pass.

It is known to combat lice and their nits using neurotoxic substances, such as pyrethrins or synthetic pyrethroids (permethrin, allethrin, bioallethrin, D-phenothrin, and the like), organophosphorus compounds, such as malathion, carbamates (carbaryl, and the like) or piperonyl butoxide. However, it appears that these substances are gradually losing their effectiveness following the appearance of strains of Pediculus humanus which are resistant to them. Furthermore, their use in the vicinity of the human body can result in skin irritation and risks due to their neurotoxicity when used repeatedly. This is because these neurotoxic substances act on the nervous system of the louse and are also toxic to man. Some of these products are even suspected of being carcinogenic, indeed even teratogenic. This toxicity is a particular concern in the case of children, for whom repeated infestations by lice often require repeated treatments.

A composition based on saccharide and on silica which is effective in combating lice is disclosed in WO03/045152. However, this composition is relatively expensive and its pediculicidal action is fairly slow.

The invention is targeted at providing a novel pediculicidal process to which the lice are not resistant and which is economic and harmless to man.

Consequently, the invention relates to a pediculicidal process which makes it possible to combat the growth of the louse Pediculus humanus, according to which the louse is brought into contact with a composition comprising at least 60% by weight of alkali metal bicarbonate.

In the process according to the invention, the alkali metal bicarbonate can, for example, be bicarbonate in the strict sense, such as potassium bicarbonate or sodium bicarbonate. In this account, however, it also covers compound salts, such as alkali metal sesquicarbonates (for example trona), which comprise bicarbonate. Sodium bicarbonate, potassium bicarbonate or trona are especially well suited. Bicarbonates in the strict sense are recommended. Potassium bicarbonate or sodium bicarbonate, more particularly sodium bicarbonate, is preferred. The composition comprises at least 60%, advantageously 70%, preferably 80%, more preferably still 85%, by weight of alkali metal bicarbonate.

The bicarbonate advantageously comprises particles having a particle size distribution such that at least 50%, advantageously 75%, preferably 90%, of the particles have a mean diameter of less than 100 μm, preferably 70 μm. The mean diameter is advantageously less than 50 μm, preferably 40 μm. The diameters are measured according to Standard ASTM C-690-1992. The remainder of the bicarbonate, which is not in the form of particles, can then, for example, be in the dissolved form. In addition to its chemical action against Pediculus humanus, the inventors consider that the alkali metal bicarbonate has a mechanical action which is more effective when it has such particle sizes.

In a preferred embodiment of the process according to the invention, the composition additionally comprises at least 1%, preferably 5%, of silica. It is recommended that it should not comprise more than 20%, preferably 15%, thereof. It has been observed that the presence of silica in the powder synergistically increases the pediculicidal effects of the alkali metal bicarbonate. Furthermore, as the powder comprises high concentrations of bicarbonate, the presence of silica improves the flow of the suspension and thus favours its homogeneous application. It is recommended that the silica should be in the amorphous (and noncrystalline) form in order to be tolerated by the human body, in particular in the event of inhalation.

It is preferable for the silica to be in the form of very fine particles having a high specific surface, for example of greater than 200 m²/g, preferably 400 m²/g, measured according to Standard ISO 5794-1, annex D. In an advantageous embodiment of the invention, the silica is in the form of particles having a mean diameter of less than 10 μm. The mean diameter is measured according to Standard ASTM C-690-1992.

The bicarbonate/silica mixture must be as homogeneous as possible. However, it has been observed that, in certain circumstances, in particular when the mixing is carried out in ploughshare mixers, which is advantageous, an excessively long mixing time can result in a loss in effectiveness of the subsequent aqueous suspension. Generally, it is recommended to handle the alkali metal bicarbonate/silica mixture so as to fluidize it. This fluidization takes place in a ploughshare mixer when the mixture falls back in the mixer following the rotation of the ploughshare.

In particular when it is in the form of particles having the most advantageous particle sizes, the bicarbonate has its own parasiticidal action and its presence in the composition does not have a function of diluting or carrying the silica.

Alkali metal bicarbonates and silica are substances harmless to man and animals. In the case of sodium bicarbonate and potassium bicarbonate, they are even permitted by various bodies (such as the Food and Drug Administration in the United States and the EEC) in human food. In the process according to the invention, the powder does not require any other substance active against Pediculus humanus, such as neurotoxic pediculicides. It is recommended that it should be devoid of any neurotoxic substance. It is even preferable for it to be completely devoid of any other active pediculicidal substance.

In the process according to the invention, the composition can be in various formulation forms, such as powder, cream, gel, aqueous suspension or aqueous solution.

In a first alternative form of the process according to the invention, the composition is in the form of an aqueous suspension. This formulation faith is advantageous for the formulating of shampoos. In this alternative form, it is recommended for the aqueous suspension, which may not be confused with an aqueous solution, to comprise an amount of alkali metal bicarbonate which exceeds its solubility limit at ambient temperature. It has been observed that, in this case, the particle size of the bicarbonate particles in the suspension is more stable over time.

In a second alternative form of the process according to the invention, which is preferred, the composition is in the gel form. Gels are compositions comprising at least two components, generally a solid dispersed in the colloidal form in a liquid phase. The dispersed particles faun spatial networks stabilized by means of Van der Waals' forces. In hydrogels, the liquid phase is water. They can also comprise gelling polymers, such as polysaccharides, pectin, xanthan, and the like. In this alternative form, it is preferable for the composition to comprise silica. When the composition comprises silica, it is particularly easy to produce a gel by simple addition of water to the bicarbonate/silica mixture. It is then preferable for the composition to be devoid of gelling polymers. This alternative form can be easily employed in particular on human head hair as prolonged contact between Pediculus humanus and the composition is provided by the consistency of the gel, which makes it possible to avoid recourse to coverings, such as caps. Finally, such a composition is completely harmless, which is advantageous in treating the lice present on young children as the latter frequently touch their head hair and may subsequently bring their hand to their mouth.

The invention thus also relates to a composition in the gel form comprising from 35 to 55%, preferably from 40 to 50%, by weight of alkali metal bicarbonate particles, from 10 to 30%, preferably from 15 to 25%, of silica and from 30 to 50%, preferably from 35 to 45%, of water.

It is advantageous for the composition not to comprise more than 10%, preferably 5%, in particular 1%, of organic matter, such as a gelling polymer, for example xanthan gum. Although in some cases it is recommended for it to comprise at least 0.1% by weight of such a gelling agent (for example xanthan gum) in order to improve the rheological properties of the gel, it is particularly advantageous for the composition to be completely inorganic.

The composition in the gel form according to the invention can be obtained very simply by simple mixing of the constituents.

Such a gel, by virtue of its simplicity of use and of its consistency, has numerous applications. It has appeared particularly useful in combating parasites in general (such as, for example, ticks, lice, acarids) as parasiticidal composition. However, it is particularly suitable for combating Pediculus humanus as pediculicidal composition by virtue of its harmlessness to man and its excellent effectiveness against the nits of this parasite. The pediculicidal composition is then advantageously devoid of any other pediculicidal substance. Without wishing to be committed to a theoretical explanation, the inventors suspect that the composition according to the invention results in blocking of the aeropiles, which prevents the nymphs from hatching.

The ingredients of the composition according to the invention are preferably, insofar as possible, in accordance with those of the composition used in the specific alternative forms and embodiments of the process according to the invention. For example, the particulate bicarbonate, which is advantageously sodium bicarbonate, is preferably in the form of particles having a particle size such that at least 50%, advantageously 75%, preferably 90%, of the particles have a mean diameter of less than 100 μm, preferably 70 μm. The gel then has a very smooth consistency which is particularly pleasant to the touch and its parasiticidal effectiveness in general but in particular its pediculicidal effectiveness is thereby improved.

The composition and the process according to the invention are particularly effective in combating Pediculus humanus infesting substrates such as human body linen, bedding and in particular pillows, cleaning materials, such as towels, human hair in general and in particular human head hair. As the composition and the process according to the invention employ nontoxic substances with a rather mild action, they are advantageously intended to combat infestations from the beginning of their development, in particular on human head hair. When they are applied to human hair, a cosmetic composition and a cosmetic process are thus advantageously concerned, the doses of active product being insufficient to prevent or treat any attack on the skin which would be due to a highly advanced infestation by lice.

Consequently, the invention also relates to a cosmetic process for eliminating the louse Pediculus humanus from human head hair, according to which the head hair is brought into contact with the composition in accordance with the invention.

In an advantageous alternative form of this process, the head hair is brought into contact with the composition for at least 1 hour, preferably 4 hours, more preferably 8 hours, the operation of bringing into contact for at least 1 hour, preferably 4 hours, more preferably 8 hours, subsequently advantageously being repeated at the earliest 2 days later. However, it is recommended for the second operation of bringing into contact to take place less than 10 days, preferably one week, after the first. Additional operations of bringing into contact may be provided, if necessary. Care will particularly be taken that the composition is applied homogeneously to the hair while definitely reaching the base of the head hair, the nits generally being attached to the part of the individual head hair closest to the scalp.

The following examples serve to illustrate the invention.

### EXAMPLE 1

Strains of the louse Pediculus humanus suited to the rabbit were selected. Right from their birth, the young hatched lice, placed on pieces of material simulating the hair, were set down once daily on the shaven stomach of a rabbit in order to gorge themselves with blood. Right after they had finished feeding, they were placed in an oven maintained at 29° C. and 70-80% relative humidity. Four batches of 25 lice aged between 3 and 7 days after hatching (nymphs), on the one hand, and aged between 15 and 16 days after hatching (adults), on the other hand, were then brought into contact for 1 hour with a powder comprising 85% by weight of sodium bicarbonate Bicar® 0/4, composed of particles having a particle size such that 75% of the particles have a mean diameter of less than 65 μm, and 15% by weight of silica Sipernat® 50S manufactured by Degussa. After the treatment, the lice were washed. 16 hours after the treatment, all the lice fed and stored normally were dead, whether nymphs or adults. In comparison, lice only washed, not having been brought into contact with the powder, showed a mortality of approximately 9%.

### EXAMPLE 2

In Example 2, two batches of 50 lice, treated in the same way as in Example 1, were brought in contact for 3 h with a gel consisting of 43% of bicarbonate (composed of particles having a particle size such that 75% of the particles have a mean diameter of less than 65 μm), 19% of silica Sipernat®50S and 38% water. Already 1 h after the treatment, 98% of the lice were dead. When the contact time was increased from 3 h to 6 h, 1 h after the treatment, 100% of the lice were dead.

### EXAMPLE 3

In example 3, lice nits, laid between 1 and 4 days previously, were taken. The nits were brought into contact for 8 hours with a powder analogous to that of Example 1. The nits were subsequently washed and placed in the oven (29° C., 70-80% relative humidity), and observed for 15 days. Only 18% of them hatched. In comparison, 92% of the nits only washed (not having been subjected to the treatments) hatched.

### EXAMPLE 4

In example 4, it has been proceeded as in example 3 except that the nits were brought into contact for 3 hours with a gel analogous to that of Example 2. Only 4% of the nits hatched and furthermore none of them survived. When the contact time was increased from 3 to 6 h, none of them hatched.

1. A pediculicidal process which makes it possible to combat the growth of the louse Pediculus humanus, according to which the louse is brought into contact with a composition comprising at least 60% by weight of alkali metal bicarbonate.

2. The process according to claim 1, in which the bicarbonate is sodium bicarbonate.

3. The process according to claim 1, in which the particulate bicarbonate is in the form of particles having a particle size such that at least 90% of the said particles have a mean diameter of less than 100 μm.

4. The process according to claim 3, in which the diameter is less than 70 μm.

5. The process according to claim 1, in which the composition additionally comprises from 5 to 20% of silica.

6. The process according to claim 5, in which the silica is in the form of particles having a mean diameter of less than 10 μm.

7. The process according to claim 1, in which the composition is in the form of an aqueous suspension.

8. The process according to claim 1, in which the composition is in the gel form.

9. A composition in the gel form, comprising from 40 to 50% by weight of alkali metal bicarbonate particles, from 15 to 25% by weight of silica and from 35 to 45% of water.

10. The composition according to claim 9, composed solely of alkali metal bicarbonate, of water and of silica.

11. A pediculicidal composition in accordance with the composition according to claim 9, devoid of any other pediculicidal substance.

12. A parasiticidal process, according to which the parasite is brought into contact with a composition according to claim 9.

13. A process for eliminating the louse Pediculus humanus from human head hair, according to which the head hair is brought into contact for at least 1 hour, preferably 4 hours, more preferably 8 hours, with a composition in accordance with claim 9, the operation of bringing into contact for at least 1 hour, preferably 4 hours, more preferably 8 hours, subsequently being repeated at the earliest two days later.
